(19) United States
(12) Patent Application Publication
(10) Pub. No.: US 2007/0134340 A1
(43) Pub. Date: Jun. 14, 2007

(54) Method for delivering hydrophobic drugs via nanocrystal formulations

(76) Inventors: Paras N. Prasad, Williamsville, NY (US); Haridas E. Pudavar, N. Tonawanda, NY (US); Koichi Baba, Sendai (JP); Indrajit Roy, Amherst, NY (US); Tymish Ohulchanskyy, Kenmore, NY (US); Ravindra K. Pandey, Williamsville, NY (US); Allan Oseroff, Buffalo, NY (US)

Correspondence Address: HODGSON RUSS LLP, ONE M & T PLAZA, SUITE 2000, BUFFALO, NY, 14203-2391, US

(21) Appl. No.: 11/471,075

(22) Filed: Jun. 20, 2006

Related U.S. Application Data
(60) Provisional application No. 60/692,145, filed on Jun. 20, 2005.

(51) Int. Cl. A61K 31/555; A61K 31/409; A61K 9/14

(52) U.S. Cl. 424/489; 514/185; 514/410; 977/906

(57) ABSTRACT

This invention provides nanocrystals or polymer doped nanocrystals of hydrophobic drug molecules as stably dispersed in an aqueous system which are prepared without stabilizers like surfactants and the like. In one embodiment, the drug is a tetra-pyrrole compound. An example is the hydrophobic photosensitizing anticancer drug 2-devinyl-2-(1-hexyloxyethyl)pyropheophorbide (HPPH). Pharmaceutical compositions comprising nanocrystals or polymer doped nanocrystals of hydrophobic drugs can be used for therapeutic purposes. For example pyropheophorbides such as HPPH can be used for photodynamic therapy. Drug efficacy of these nanocrystals were found to be comparable with that of same drug formulated in conventional delivery vehicles under in vitro and in vivo conditions.

This application claims priority to U.S. Provisional Application No. 60/692,145 filed on Jun. 20, 2005, the disclosure of which is incorporated herein by reference.

This invention was made with funds from United States Air Force/AFOSR Grant no. F49620-0101-0358. The Government has certain rights in the invention.

### FIELD OF THE INVENTION

The present invention relates generally to the field of cancer and more particularly provides an efficient method for the use of hydrophobic drugs for photodynamic therapy.

### DISCUSSION OF RELATED ART

Photodynamic therapy (PDT) is an emerging modality for the treatment of a variety of oncological, cardiovascular, dermatological and ophthalmic diseases [1]. PDT is based on the concept that light sensitive species or photosensitizers (PS) can be preferentially localized in tumor tissues upon systemic administration [2,3]. When such photosensitizers are irradiated with an appropriate wavelength of visible or near infra-red (NIR) light, the excited molecules can transfer their energy to molecular oxygen in the surrounding. This results in the formation of reactive oxygen species (ROS), like singlet oxygen (¹O₂) or free radicals. These locally generated ROS are responsible for oxidizing various cellular compartments including plasma, mitochondria, lysosomal and nuclear membranes etc., resulting in irreversible damage of tumor cells [2-6]. Therefore, under appropriate conditions, PDT offers the advantage of an effective and selective method of destroying diseased tissues without damaging adjacent healthy ones.

Most of the commonly used photosensitizing drugs (PS) are poorly water soluble or hydrophobic, and therefore, preparation of pharmaceutical formulations for parenteral administration is highly hampered [2,7]. To overcome this difficulty, different strategies have evolved to enable the stable dispersion of these drugs into aqueous systems, often by means of a delivery vehicle. Upon systemic administration, such drug-doped carriers are preferentially taken up by tumor tissues by the virtue of the ‘Enhanced Permeability and Retention Effect” [2, 8-11] which is the property of such tissues to engulf and retain circulating macromolecules and particles owing to their ‘leaky’ vasculature. The carriers include oil-dispersions (micelles), liposomes, polymeric micelles, hydrophilic drug-polymer complexes, etc. Oil-based drug formulations (micellar systems) using non-ionic poloxyethylated castor oils (e.g. Tween-80, Cremophor-EL or CRM etc.) have shown enhanced drug loading and improved tumor uptake over free drugs, presumably due to interaction with plasma lipoproteins in blood [12,13]. However, such emulsifying agents also have been reported to elicit acute hypersensibility (anaphylactic) reactions in vivo [9, 10]. Liposomes are concentric phospholipid bilayers encapsulating aqueous compartments, which can contain hydrophilic and lipophilic drugs [2]. Although the tumor uptake of liposomal formulation of drugs is better than that of simple aqueous dispersions, many suffer from poor drug loading and increased self-aggregation of the drug in the entrapped state [2, 11]. Liposomes are also prone to opsonization and subsequent capture by the major defense system of the body (reticulo-endothelial system, or RES). Recently, drugs incorporated inside pH sensitive polymeric micelles have shown improved tumor phototoxicity compared to CRM formulations in vitro, however in-vivo studies showed poor tumor regression and increased accumulation in normal tissues [4, 12]. Recently another approach using drug encapsulated nanoparticles made of organically modified silica particles (ORMOSIL) as carrier for PDT drugs had been proposed [14].

In all these above formulations, external stabilizing agents such as surfactants (e.g., Tween 80) or polymeric micelles (e.g., Phospholipid-PEG) or other organic or inorganic matrices (e.g., silica) are used along with the photosensitizer. Although the stable aqueous dispersion of the photosensitizer is facilitated using these stabilizers, a number of drawbacks for effective tumor-specific drug delivery are also introduced. Firstly, as the pharmacodynamics of the drug/carrier composite largely depends on the nature of the carrier matrix, the intrinsic tumor-avidity of the photosensitizers are not fully exploited. Even with the tumor-associated EPR effect, only a fraction of the delivered photosensitizers reach the intended target. Although this poor tumor-specific delivery can be substantially improved with the use of targeting molecules like certain peptides and antibodies, the formulations become highly immunogenic owing to their presence of proteins at the surface. Therefore it is critical to limit the number of components to be used in the formulation of a nanosized drug-delivery carrier and develop methods for delivering PDT drugs, which are free from external agents.

### SUMMARY OF THE INVENTION

In the present invention, nanocrystals of hydrophobic drug molecules such as tetra-pyrrole compounds have been synthesized which remain stably dispersed in an aqueous system without the necessity of stabilizers like surfactants. In one embodiment, nanocrystals of a hydrophobic photosensitizing anticancer drug 2-devinyl-2-(1 -hexyloxyethyl) pyropheophorbide (HPPH), were synthesized using reprecipitation method. In a variation of the above method, polymer doped nanocrystals of hydrophobic drugs can also be used.

The compositions comprising nanocrystals or polymer doped nanocrystals of hydrophobic drugs can be used for therapeutic purposes. For example tetra-pyrrole compounds including, but not limited to, pyropheophorbides such as HPPH, can be used for photodynamic therapy. Though the fluorescence and photodynamic activity of the drug nanocrystals were substantially quenched in aqueous media, both recovered inside cells or under in vivo conditions. Drug efficacy of these nanocrystals were found to be comparable with that of same drug formulated in conventional delivery vehicles under in vitro and in vivo conditions

### BRIEF DESCRIPTION OF THE DRAWINGS

FIG. 1. Sturcture of the drug HPPH

FIG. 2. TEM picture of HPPH nanocrystals

FIG. 3. Absorption and emission spectra of HPPH nanocrystals (4 μM) in comparison with Tween80 micellar formulation at the same molar concentration in water.

FIG. 4. Fluorescence signal from HPPH nanocrystals (4 μM) in culture media and in presence of FBS or BSA after 6 hrs of incubation at room temperature.

FIG. 5. Fluorescence signal recovery from HPPH nanocrystals (4 μM) in culture media and in presence of FBS and BSA.

FIG. 6. Singlet oxygen phosphorescence spectra of D₂O dispersion of HPPH nanocrystals and similar concentration of 1% Tween80 micellar formulation in D₂O. Absorbance was matched at the wavelength of excitation.

FIG. 7. Confocal fluorescence images of HPPH nanocrystals (A) and Tween80 micellar formulation (B) stained HeLa cells imaged after 2 hrs of incubation in serum free media. Cells were incubated with 100 μl of 60 μM formulation in both cases. Excitaiton wavelength used was 420 nm.

FIG. 8. Comparative in vitro photosensitizing efficacy of HPPH formulated in 1% Tween 80/5% dextrose and HPPH nanocrystals/water in RIF cells at equimolar concentrations (0.5 μM). Control: Cells were incubated with photosensitizers but no light exposure.

FIG. 9 Comparison of in vivo photosensitizing efficacy of HPPH nanocrystals and Tween80 micellar formulation in C3H mice (5 mice/group) bearing RIF tumors. The tumors were exposed to a laser light (665 nm, 135 J/cm2) at 24 h after injection.

### DETAILED DESCRIPTION OF THE INVENTION

In the present invention, the use of aqueous dispersions of hydrophobic drug nanocrystals or polymer doped nanocrystals is described. Accordingly, this invention provides compositions and methods for the delivery of hydrophobic drugs prepared without the use of stabilizing agents such as surfactants or other carriers or delivery vehicles. The resulting drug nanocrystals or polymer doped nanocrystals are monodispersed, and stable in aqueous system.

The drug nanocrystals are avidly taken up by cancer cells, as shown by confocal microscopy. Though the fluorescence and photodynamic activity of the drug nanocrystals were substantially quenched in aqueous media, both were surprisingly observed to recover inside cells or under in vivo conditions. While not intending to be bound by any particular theory, it is considered that the recovery of fluorescence is attributable to drug-Serum Albumin interaction. This is based upon similar recovery in presence of Fetal Bovine Serum (FBS) or Bovine Serum Albumin (BSA). Thus, upon administration for PDT use in humans, it is believed that the drug will interact with human serum albumin or other intracellular or extracellular molecules for allowing the recovery of fluorescence. Drug efficacy of these nanocrystals were found to be comparable with that of same drug formulated in conventional delivery vehicles under in vitro as well as in vivo conditions.

These nanocrystals are uniform in size distribution with an average size generally less than 150 nm, preferably less than about 120 nm, more preferably less than 100 nm. In one embodiment, the average size is less than 50 nm and in another embodiment, the average size is between 30 to 40 nm. The nanocrystals can be formulated as a stable aqueous dispersion. Such nanocrystals are efficiently taken up by tumor cells, in vitro. Light irradiation of such impregnated cells resulted in significant cell-death. In-vivo study of these drug nanocrystals also showed significant efficacy equivalent to the conventional surfactant based delivery mechanism. These observations have identified the potential of using surfactant free drug nanocrystals for PDT. This approach eliminates the need of any external agents such as surfactants or other carrier matrices for drug delivery. Potentially this method of drug delivery can be applied not only for PDT drugs, but also for delivery of other therapeutic drugs including chemotherapeutic drugs.

The nanocrystals of the hydrophobic dye are generated by a simple two step method, comprising of dissolving millimolar amounts of a hydrophobic drug in an organic solvent and then mixing a small amount of the organic solution with water to form the drug nanocrystals. It has been shown by X-ray diffraction measurements that such structures are crystalline in nature [19].

In one variation of the above method, polymer doped drug nanocrystals are generated which exhibit reduced initial quenching of fluorescence in a comparison to the undoped ones. For preparation of polymer doped drug nanocrystals, the drug and polymer are dissolved in the organic solvent and the solution is then mixed with water to form the polymer doped nanocrystals. The percentage of polymer in the polymer doped nanocrystals is less than 20% or 10%. In a preferred embodiment, the polymer is less than 10%. Examples of polymers that can be used include but not limited to polylactides (PLA), Polyglycolides (PGA), Poly(lactide-co-glycolides) (PLGA), coplymers thereof, polyanhydrides polyorthoesters, and polysiloxylanes.

To prepare the nanocrystals, the drug is dissolved in an organic solvent which is water miscible. Non-limiting examples of water miscible organic solvents include, but are not limited to, acetone, ethanol and dimethyl sulfoxide (DMSO), dimethylformamide (DHF), tetrahydrofuran (THF), ethyl acetate,methyl iso-butyl ketone, methyl acetate, methyl propyl ketone, iso-pentyl alcohol, iso-propyl alcohol, methyl alcohol, ethylene glycol monobutyl ether and propylene glycol monomethyl ether. Solvents which are not miscible with water i.e., hexane, chloroform and benzene are not suitable. The size of the nanocrystals or polymer doped nanocrystals can be varied by controlling various factors. For example the concentration of the drug in the organic solvent is an important factor. Size of the nanocrystals increases with increase in drug concentration and it can be adjusted from few tens of nanometers to few hundreds of nanometers, by tuning the concentration. Further, it is preferable to use water at a temperature such that a uniform distribution of the nanocrystals is obtained and there is no aggregation of the dye nanocrystals. Another factor is the ratio of the organic solvent to water. The volume of the organic solvent added to water is preferably about 10-100 times less than the volume of the water. For example, the volume of DMSO solution (containing the drug) is preferably about 200 μl in about 10 ml of water.

The hydrophobic drug is dissolved in an organic solvent selected based on the drug solubility at millimolar concentration (ranging from 1 mM-100 mM based on drug solubility in the selected organic solvent). After preparation of the solution of the objective drug, microliter quantities of this solution are added to water. To get a homogenous population of nanocrystals, it is preferable to add the drug-solvent solution quickly into water with continuous stirring. For example, the drug-solvent solution can be added to water using a mircosyringe while the water is magnetically stirred (at about 1000 rpm). After this two step procedure, an aqueous dispersion of nanocrystals is obtained. While not intending to be bound by any particular theory, it is believed that as the surface of the nanocrystals is negatively charged (as determined by zeta potetntial measurements of few tens of mV), the dispersion is electrostatically stabilized to form water dispersions without aggregation.

The solvent can be removed from the preparation by standard methods such as ultracentrifugation or dialysis after fabrication of nanocrystals. Dialysis can be carried out by routine methods, using a dialysis membrane of cut-off size 3.5 kD and above.

This method is applicable to hydrophobic drugs in general. In one embodiment, tetra-pyrrole compounds are used. The compounds are represented by the following structure:

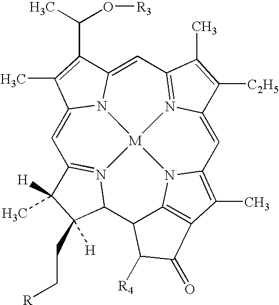

wherein R═COOH, COOR₁ (where R₁=various alkyl groups), or R═CONHR₂ (where R₂═various peptides and aminoacids), R3=alkyl groups with 1 to 12 carbon units, aryl and substituted aryl substituted alkyl, R4=H or —C═O, M=2H or various metals (In, Zn, Mg, Pd etc.); or

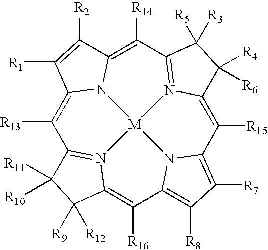

wherein: R₁-R₁₆═H, M=2H or various metals [In, Zn, Mg, Pd etc.], R₃, R₄, R₉, R₁₀═OH, R₁₃-R16═H or aromatic, substituted aromatic, alkyl, ester substituted ester with amide bond etc., R₅, R₆, R₁₁ and R₁₂═H or folded to a bond between the carbon attached to R₅, R₆ and R₁₁ and R₁₂, or

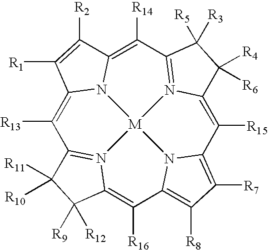

wherein: R═COOH, COORₓ (where Rₓ=various alkyl groups), R═CONHR₂ (where R₂=various peptides and aminoacids), R₁=alkyl groups with 1 to 12 carbon units, aryl and substituted aryl substituted alkyl, R₆=Open chain or closed five-, substituted five, six member substituted six member ring system, R₂, R₃═H or OH, or folded to a bond between the attached pyrrolic carbon units, R₄ and R₅═H or OH or folded to a bond between the attached carbon units, M=2H, In, Zn, Pd etc.

One class of tetra-pyrrole compounds is the pyropheophorbide compounds which are very useful for PDT. Several examples of such compounds are presented in U.S. Pat. No. RE39,094, the disclosure of which is incorporated herein by reference.

The nanocrystal or polymer doped nanocrystal compositions can be administered to animals including humans in therapeutically effective amounts by any standard route. For example, the compositions may be administered by intravenous, intramuscular or intradermal routes. These compounds are known to selectively accumulate in tumor cells. After sufficient time following administration so that the drug accumulates in tumor cells, the cells are exposed to an appropriate wavelength of light which causes the compound to become cytotoxic thereby selectively destroying the tumor cells.

The following examples are provided for the pyropheophorbide, HPPH. However, those skilled in the art will recognize that the method described herein can be used for any other tetra-pyrrole drugs as described herein.

### EXAMPLE 1

This example describes the synthesis and characterization of the nanocrystals. Reprecipitation method was used to prepare aqueous suspension of the drug as organic nanocrystals. For this, 200 μl of 3 mM HPPH solution in DMSO was injected into 10 ml of water at room temperature, with controlled stirring. The samples were dialyzed overnight to remove organic solvents. The size of particle was controlled by tuning the initial concentration of dye in DMSO solution. The size of nanoparticle was estimated as about 100 nm. Transmission electron microscopy (TEM) was employed to determine the morphology and size of the aqueous dispersion of nanocrystals, using a JEOL JEM 2020 electron microscope, operating at an accelerating voltage of 200 kV. UV-visible absorption spectra were recorded using a Shimadzu UV-3101 PC spectrophotometer, in a quartz cuvette with 1 cm path length. Fluorescence spectra were recorded on a Shimadzu RF 5301U spectrofluorimeter.

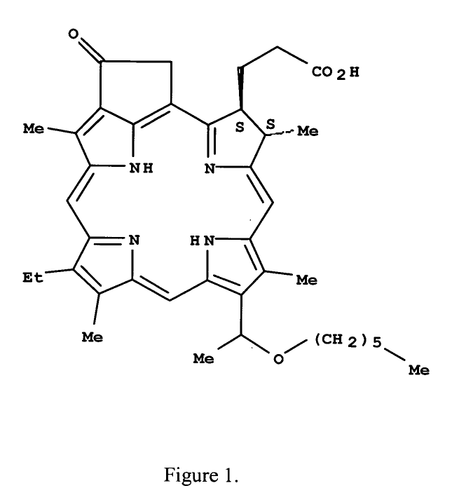

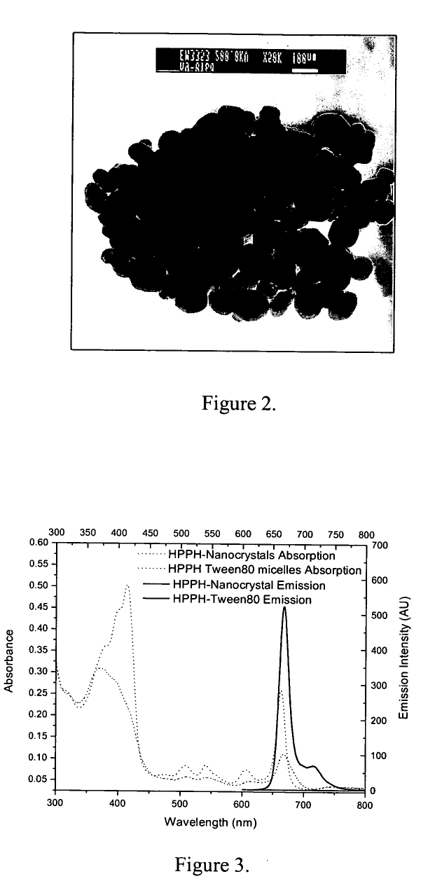

The structure of the hydrophobic drug HPPH (a chlorophyll-a analog) used in this study is shown in FIG. 1. Previously this method has been demonstrated to generate crystalline structures of compounds [19]. A TEM image of the nanocrystals of the drug HPPH prepared by reprecipitaiton method is shown in FIG. 2. The particles are nearly spherical, having uniform size distribution, with an average size of 110 nanometers. Dynamic light scattering measurements (DLS) also showed reasonably good monodispersity with size ranging from 100-120 nm.

The UV-visible absorption and fluorescence emission spectra (excitation wavelength=532 nm) of HPPH formulated as nanocrystals show significant effects of molecular aggregation in nanocrystals, and can be easily distinguished from the same spectra obtained with HPPH dissolved in 1% Tween-80/water. This includes suppression of Soret band and broadening of the longwave Q-band in the absorption spectra as well as almost complete quenching of the HPPH monomer emission (FIG. 3). The latter is well known for aggregation of fluorescent molecules [15]. This quenching of fluorescence of aggregated dye molecules is well understood. Typically, this fluorescence quenching in case of PDT drugs is correlated with reduced ROS generation and reduced efficacy. But our study indicate that in case of the drug HPPH and potentially in case of many other drugs, the fluorescence as well as PDT efficacy (due to the generation of singlet oxygen) of nanocrystals formulation recovers under in vitro and in vivo conditions.

### EXAMPLE 2

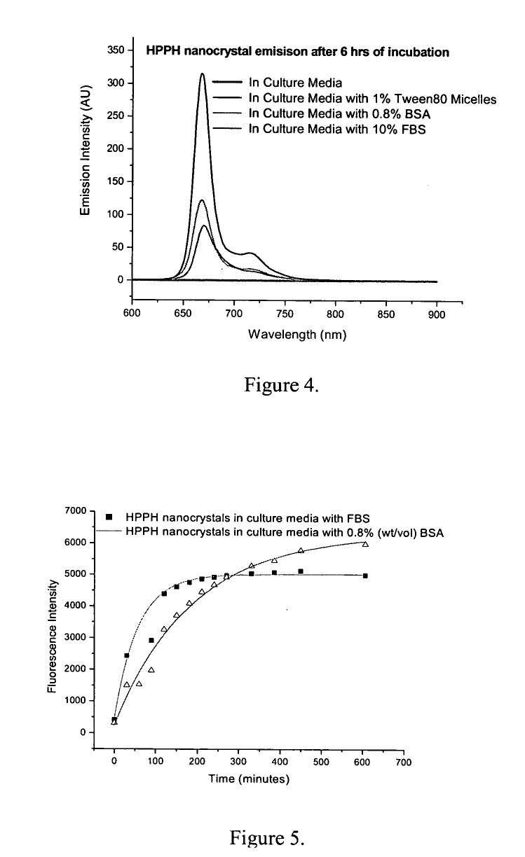

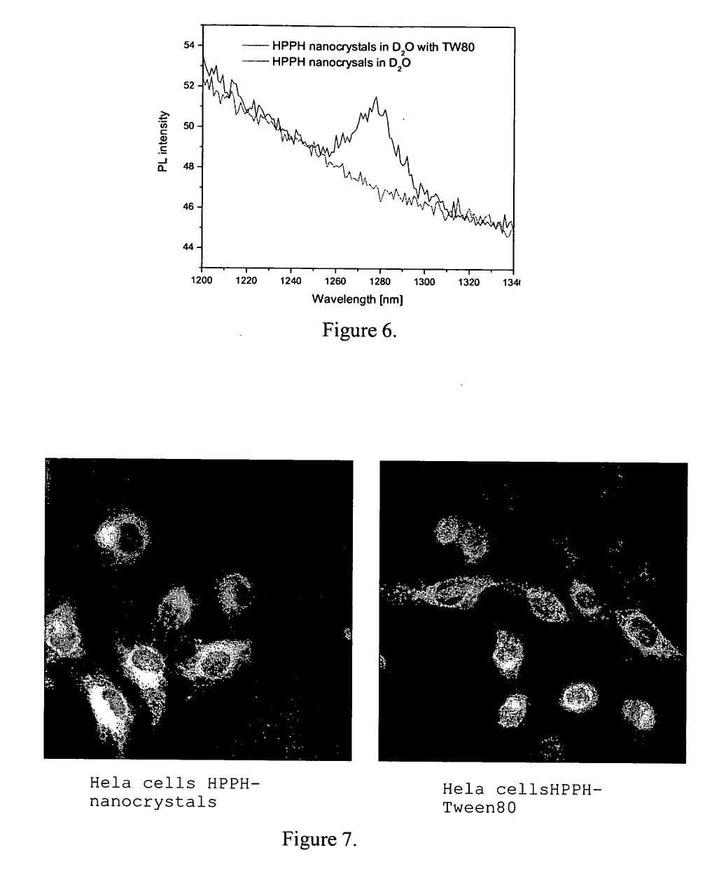

This example describes the recovery of quenched fluorescence as well as the ability to generate Singlet oxygen (one of the main Reactive Oxygen Species causing cellular phototoxicity) under simulated in vitro or in vivo conditions. As seen from FIG. 5, fluorescence spectra of HPPH were quenched in the nanocrystals formulation, in comparison with the Tween80 micellar preparation of the same concentration. For mimicking the in vitro cell culture conditions 10% FBS was added to the HPPH nanocrystals suspensions and fluorescence from HPPH nanocrystals was found to be increasing in a time dependent manner. This recovery of fluorescence was attributed to the presence of Bovine Serum Albumin (BSA) and other lipoproteins in the serum. To test this idea we also added 08% (wt/vol) of BSA to HPPH nanocrystal suspension and again the fluorescence was found to be recovering. FIG. 4 shows the emission spectra of the nanocrystals formulation after 4.5 hrs of incubation with 10% FBS as well as 0.8% (Wt/Volume) BSA. The time-course of the fluorescence recovery in presence of BSA and FBS is shown in FIG. 5.

Phosphorescence spectroscopy method was used for detection of singlet oxygen. Along with fluorescence quenching, the singlet oxygen generation also was quenched in this nanocrystals geometry as seen from the singlet oxygen phosphorescence spectra shown in FIG. 4. Detection of singlet oxygen (¹O₂) has been extensively reported by its phosphorescence emission spectra at 1270 nm [16, 17]. We have used deuterium oxide (D₂O) as a solvent because it extends the lifetime of singlet oxygen compared to water A SPEX 270M Spectrometer (Jobin Yvon) equipped with a Hamamatsu IR-PMT was used for recording singlet oxygen phosphorescence spectra. A 514 nm laser line from an Argon laser (Spectra Physics) was used as the excitation source. The sample solution in a quartz cuvette was placed directly in front of the entrance slit of the spectrometer and the emission signal was collected at 90-degrees relative to the exciting laser beam. An additional longpass filters (a 950LP filter and a 538AELP filter, both from Omega Optical) were used to attenuate the excitation laser and the fluorescence from HPPH.

Under in-vitro conditions, in presence of Fetal Bovine Serum (FBS) or Bovine Serum Albumin (BSA), the singlet oxygen generation was also found to be recovering in a time-dependent manner.

### EXAMPLE 3

This examples described the in-vitro studies with tumor cells and nanoparticle uptake, imaging and viability assay.

Cell Culture. Human cervical carcinoma cell line (HeLa) was maintained in Dulbecco's modified eagle medium with 10% FBS according to the manufacturers instructions (American Type Culture Collection, Manassas, Va.). To study the uptake and imaging of HPPH nanocrystals, the cells were trypsinized and resuspended in the corresponding suitable media at a concentration of around 7.5×10⁵/ml. 60 μl of this suspension was transferred to each 35 mm culture plate and 2 ml of the corresponding full medium was added. These plates were then placed in an incubator at 37° C. with 5% CO₂ (VWR Scientific, model 2400). After 36 hours of incubation, the cells (about 60% confluency) were rinsed with PBS, and 2 ml of the corresponding fresh media was added to the plates. Finally, 50 μl of HPPH nanocrystals was added and mixed properly. Plates were returned to the incubator (37° C., 5% CO₂) for the required incubation period. 50 μl of HPPH/Tween80 micelles of same drug concentration was used as control and separate culture plates were treated and incubated for the same time period as in the case of nanocrystals. After each specific time interval of incubation, the plates were taken out, rinsed several times with sterile PBS and 2 ml of fresh serum free medium was added. The plates were incubated for another 10 minutes at 37° C. and were directly imaged under confocal a microscope as follows.

Confocal Microscopy: Confocal imaging was performed using a laser scanning confocal microscope (MRC-1024, Bio-Rad, Richmond, Calif.), which was attached to an upright microscope (Nikon model Eclipse E800). A water immersion objective lens (Nikon, Fluor-60X, NA 1.0) was used for cell imaging. For this, the Ti:Sapphire laser(Tsunami from Spectra Physics pumped by a diode pumped solid state laser, Millenia, also from Spectra Physics), tuned to 840 nm(˜100 fs pulses at 82 MHz), was doubled by Second Harmonic Generation (SHG) in a beta barium borate(β-BBO) crystal to obtain the 420 nm light, and was coupled into a single mode fiber for delivery into the confocal scan head. A combination of long pass and short pass filters were used to cut off the excitation light from entering the detection channels (Long pass filter 585LP and band pass filter BP680/30, both from Chroma technology Corp.). Cells untreated with the drugs were used as control to confirm the absence of any significant autofluorescence under the imaging conditions. In addition, to distinguish HPPH emission from, autofluorescence, we have also used localized spectrofluorometry [18]. For this purpose, the fluorescence signal was collected, without filtering, from the upper port of the confocal microscope, using a multimode optical fiber of core diameter 1 mm, and was delivered to a spectrometer (Holospec from Kaiser Optical Systems, Inc.) equipped with a cooled charge coupled device (CCD) camera (Princeton Instruments) as a detector. A comparison of fluorescence spectra from drug treated cells and fluorescence spectra of HPPH in solution allows us to confirm the origin of fluorescence seen in fluorescence image channel.

For In-Vitro fluorescence confocal imaging of nanocrystals uptake, we used fluorescence imaging to determine if HPPH nanocrystals were taken up by tumor cells. Though the fluorescence of nanocrystals were quenched in water, after cellular uptake, the fluorescence recovered in a similar fashion shown in case of BSA or FBS containing media. The fluorescence images of HeLa cells incubated with HPPH nanocrystals (FIG. 7,A) and HPPH/Tween80 micellar formulation (FIG. 7,B) show significant intracellular staining in the cytoplasm, in both cases. This indicates that cellular uptake of these nanocrystals formulations is very similar to that of Tween80 micellar formulation.

### EXAMPLE 4

This example describes the use of the drug nanocrystals for in vitro PDT. The RIF tumor cells grown in alpha-minimum essential medium (R-MEM) with 10% fetal calf serum, L-glutamine, and penicillin/streptomycin/neomycin were maintained in 5% CO2, 95% air, and 100% humidity. These cells were plated in 96-well plates at a density of 5×10³ cells/well in complete media as a means to determine PDT efficacy. The next day, photosensitizer was added at variable concentrations (1.25-20 iM). After the 24 h incubation in the dark at 37° C., the cells were replaced with fresh media and exposed to light at a dose rate of 3.2 mW/cm² at various light doses (1-20 J). The dye laser (375; Spectra Physics, Mt. View, Calif.) excited by an argon-ion laser (171 laser; Spectra-Physics, Mt. View, Calif.) was tuned to emit the drug-activating wavelength 665 nm. Uniform illumination was accomplished using a 600 μm diameter quartz optical fiber fitted with a graded index refraction lens. Following illumination, the plates were incubated at 37° C. in the dark for 48 h. Appropriate controls using identical drug doses without light irradiation (dark toxicity) were also evaluated. Following the 48 h incubation in the dark the plates were evaluated for cell viability using the MTT assay, as described below.

Cell Viability Assay. Cell viability was measured using the 3-[4,5-dimethylthiazol-2-yl]-2,5-diphenyl tetrazoliumbromide (MTT) assay. Immediately following light treatment, the cells were incubated for 48 h in the dark at 37° C. After 48 h, 10 iL of 4.0 mg/mL solution of MTT dissolved in PBS (Sigma Chemical Co., St. Louis, Mo.) was added to each well. After the 4 h MTT incubation, the MTT+ media were removed and 100 μl of dimethyl sulfoxide was added to solubilize the formazin crystals. The PDT efficacy was measured by reading the 96-well plate on a microtiter plate reader (Miles Inc., Titertek Multiscan Plus MK II) at an absorbance of 560 nm. The results were plotted as percent survival compared with the corresponding dark control (drug, no light) for each compound tested. Each data point represents the mean from a typical experiment with four replicate wells, and the error bars are the standard deviation from three separate experiments.

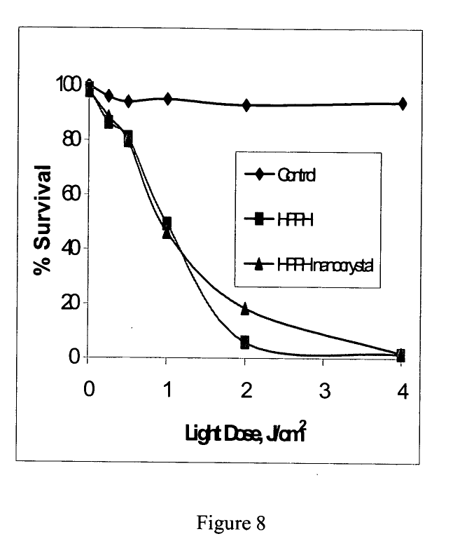

The in vitro photosensitizing efficacy was determined as follows. As seen from the FIG. 8, the in-vitro efficacy of the drug HPPH in the nanocrystals formulation as well as in the more conventional Tween80 micellar formulations showed similar light dose response. This shows the efficacy of the drug is not affected in this surfactant-free formulation.

### EXAMPLE 5

This example described the evaluation of in vivo photosensitizing efficacy. The in vivo efficacy of HPPH nanocrystals was compared with HPPH under similar treatment conditions. In brief, C3H mice (5 mice/group) were injected subcutaneously in the axilla with 3×10⁵ RIF cells in 40 μL of complete R-MEM and permitted to grow until they were 4-5 mm in diameter. The day before PDT light treatment, the mice were injected intravenously with HPPH or HPPH nanocrystals at a dose of 0.47 μmole/kg. At 24 h post-injection, the mice were restrained in plastic holders without anesthesia and treated with a laser light (665 nm, 135 J/cm², 75 mW/cm²) for 30 min and were observed daily. The tumors were measured using two orthogonal measurements L and W (perpendicular to L), and the volumes were calculated using the formula V) LW²/2 and recorded. Mice were considered cured if there was no palpable tumor by day 90.

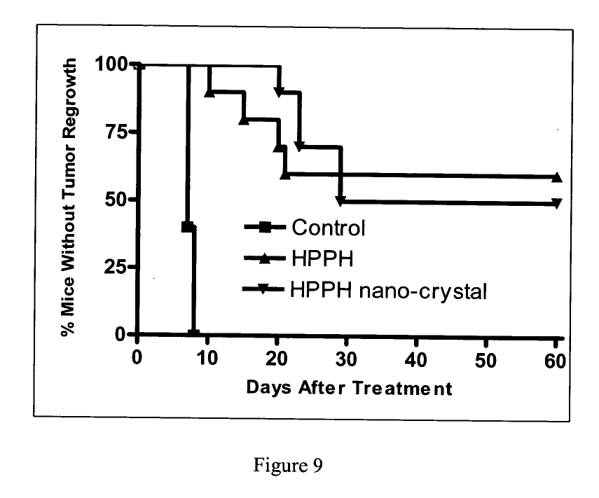

The results of the in vivo efficacy of HPPH (1% Tween 80) and the HPPH nanocrystals are summarized in FIG. 9. As can be seen, under similar treatment conditions both formulations produced a similar long-term efficacy. At day 60 (5-6/10 mice were tumor free and no visual toxicity was observed.

While the present invention has been illustrated by specific embodiment, routine modifications to the embodiments will be apparent to those skilled in the art, which modifications are intended to be within the scope of the invention.

### References

1. Levy, J. G.; Obochi, M., Photochemistry and Photobiology 1996, 64, (5), 737-739.
2. Konan, Y. N.; Gurny, R.; Allemann, E., Journal of Photochemistry and Photobiology B-Biology 2002, 66, (2), 89-106.
3 Hasan, T.; Moor, A. C. E.; Ortel, B., Photodynamic Therapy of Cancer. In Cancer Medicine 5th Edition ed.; B.C. Decker Inc.,: Hamilton, 2000.
4 Dougherty, T. J., Journal of Clinical Laser Medicine & Surgery 2002, 20, (1), 3-7.
5 MacDonald, I. J.; Morgan, J.; Bellnier, D. A.; Paszkiewicz, G. M.; Whitaker, J. E.; Litchfield, D. J.; Dougherty, T. J., Photochemistry and Photobiology 1999, 70, (5), 789-797.
6 Peng, T. I.; Chang, C. J.; Jou, S. B.; Yang, C. M.; Jou, M. J., Optical and Quantum Electronics 2005, 37, (13-15), 1377-1384.
7 Taillefer, J.; Jones, M. C.; Brasseur, N.; Van Lier, J. E.; Leroux, J. C., Journal of Pharmaceutical Sciences 2000, 89, (1), 52-62.
8 Torchilin, V. P., European Journal of Pharmaceutical Sciences 2000, 11, S81 -S91.
9 Honzak, L.; Sentjurc, M.; Swartz, H. M., Journal of Controlled Release 2000, 66, (2-3), 221-228.
10 Sobolev, A. S.; Rozenkranz, A. A.; Gilyazova, D. G., Biofizika 2004, 49, (2), 351-379.
11 Lukyanov, A. N.; Torchilin, V. P., Advanced Drug Delivery Reviews 2004, 56, (9), 1273-1289.
12 Kessel, D.; Woodburn, K., International Journal of Biochemistry 1993, 25, (10), 1377-1383.
13 Chowdhary, R. K.; Sharif, I.; Chansarkar, N.; Dolphin, D.; Ratkay, L.; Delaney, S.; Meadows, H., Journal of Pharmacy and Pharmaceutical Sciences 2003, 6, (2), 198-204.
14 Roy, I.; Ohulchanskyy, T. Y.; Pudavar, H. E.; Bergey, E. J.; Oseroff, A. R.; Morgan, J.; Dougherty, T. J.; Prasad, P. N., Journal of the American Chemical Society 2003, 125, (26), 7860-7865.
15 Yuzhakov, V. I., Uspekhi Khimii 1992, 61, (6), 1114-1141.
16 Anderson, T. M.; Dougherty, T. J.; Tan, D. F.; Sumlin, A.; Schlossin, J. M.; Kanter, P. M., Anticancer Research 2003, 23, (5A), 3713-3718.
17 Bellnier, D. A.; Greco, W. R.; Nava, H.; Loewen, G. M.; Oseroff, A. R.; Dougherty, T. J., Cancer Chemotherapy and Pharmacology 2006, 57, (1), 40-45.
18 Wang, X. P.; Pudavar, H. E.; Kapoor, R.; Krebs, L. J.; Bergey, E. J.; Liebow, C.; Prasad, P. N.; Nagy, A.; Schally, A. V., Journal of Biomedical Optics 2001, 6, (3), 319-325.
19 Kasai, H.; Nalwa, H. S.; Okada, S.; Oikawa, H.; Nakanishi, H., Handbook of Nanostructured Materials and Nanotechnology 2000, 5, 433-473.

We claim:

1. A method for effecting the destruction of a cell comprising the steps of contacting the cell with a effective amount of an aqueous dispersion of nanocrystals comprising a tetra-pyrrole compound and irradiating the cell with a light of a wavelength absorbed by the tetra-pyrrole compound thereby effecting the destruction of the cell.

2. The method of claim 1, wherein the nanocrystals comprise a tetra-pyrrole is a pyropheophorbide.

3. The method of claim 1, wherein the pheophorbide is 2-devinyl-2-(1 -hexyloxyethyl)pyropheophorbide.

4. The method of claim 1, wherein the aqueous dispersion of nanocrystals is prepared by a method comprising dissolving the tetra-pyrrole compound in a water miscible solvent to form a solution and adding the said solution into water.

5. The method of claim 4, wherein the ratio of water miscible solvent to water is about 1:10 to 1:100.

6. The method of claim 1, wherein the aqueous dispersion is substantially free of surfactants.

7. The method of claim 4, wherein the water miscible solvent is selected from the group consisting of dimethyl sulfoxide (DMSO), dimethylformamide (DHF), tetrahydrofuran (THF), ethyl acetate, methyl iso-butyl ketone, methyl acetate, methyl propyl ketone, iso-pentyl alcohol, iso-propyl alcohol, methyl alcohol, ethylene glycol monobutyl ether and propylene glycol monomethyl ether.

8. The method of claim 1, wherein the mean size of the nanocrystals is less than 150 nm.

9. The method of claim 7, wherein the mean size of the nanocrystals is less than 100 nm.

10. The method of claim 8, wherein the mean size of the nanocrystals is between 30 to 40 nm.

11. The method of claim 1, wherein the cells are contacted with an aqueous dispersion of nanocrystals which further comprise a polymer such that the concentration of the polymer in the nanocrystals is less than 20%.

12. The method of claim 11, wherein the concentration of the polymer is less than 10%.

13. The method of claim 4, wherein the nanocrystals are prepared by a method comprising simultaneously dissolving the tetrapyrrole compound and a polymer in a water miscible solvent to form a solution and adding said solution into water.

14. The method of claim 13, wherein the polymer is selected from the group consisting of Polylactides (PLA), Polyglycolides (PGA), Poly(lactide-co-glycolides) (PLGA), co-polymers thereof, Polyanhydrides, Polyorthoesters and Polysiloxylanes.

15. The method of claim 1, wherein the cells are tumor cells.

16. A pharmaceutical composition comprising nanocrystals comprising a tetra-pyrrole compound selected from the group consisting of: 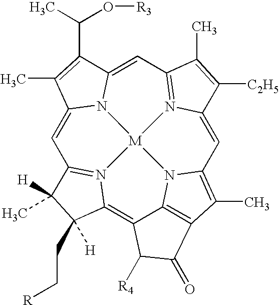
wherein R═COOH, COOR₁ (where R₁=various alkyl groups) or R═CONHR₂ (where R₂ peptides and aminoacids),
R3 alkyl group with 1 to 12 carbons, aryl and substituted aryl substituted alkyl,and
R4=H or —C═O, M=metal atom or denotes 2H1; 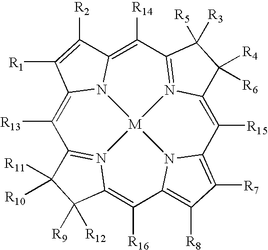
R₁-R₁₆═H, M=metal atom or denotes 2H,
R₃, R₄, R₉, R₁₀═OH,
R13-R16=H or aromatic, substituted aromatic, alkyl, ester substituted ester with amide bond,
R₅, R₆, R₁₁, and R₁₂═H or folded to a bond between the carbon attached to R₅, R₆ and R₁₁ and R₁₂; or 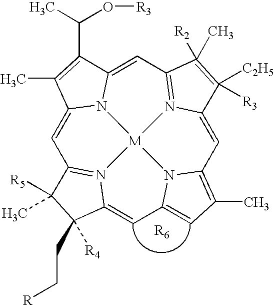
R═COOH, COORₓ (where Rₓ=alkyl group),
R═CONHR₂ (where R₂=various peptides and aminoacids),
R₁=alkyl groups with 1 to 12 carbon units, aryl and substituted aryl substituted alkyl,
R₆=Open chain or closed five-, substituted five, six member or substituted six member ring system,
R₂, R₃═H or OH, or folded to a bond between the attached pyrrolic carbon units,
R4 and R5=H or OH or folded to a bond between the attached carbon units, and
M=metal atom or denotes 2H

17. The pharmaceutical compositions of claim 12 wherein the tetra-pyrrole compound is a pyropheophorbide.

18. The pharmaceutical composition of claim 17, wherein the pyropheophorbide is 2-devinyl-2-(1-hexyloxyethyl) pyropheophorbide.

19. The pharmaceutical composition of claim 16, wherein the nanocrystals further comprise one or more polymers such that the total polymers are less than 20 wt %.

20. The pharmaceutical composition of claim 19, wherein the polymer is PLGA.
